(19) United States
(12) Patent Application Publication
(10) Pub. No.: US 2021/0386343 A1
(43) Pub. Date: Dec. 16, 2021

(54) REMOTE PREDICTION OF HUMAN NEUROPSYCHOLOGICAL STATE

(71) Applicant: SENSORITY LTD., Ashdod (IL)

(72) Inventors: Dmitry GOLDENBERG, Ashdod (IL); Katerina KON, Ashdod (IL); Elliot SPRECHER, Tel Aviv (IL); Yuval ODED, Tel Aviv (IL)

(21) Appl. No.: 17/282,926

(22) PCT Filed: Oct. 2, 2019
(86) PCT No.: PCT/IL2019/051081

§ 371 Date: Apr. 5, 2021

(30) Foreign Application Priority Data
Oct. 3, 2018 (IL) 262116

(51) Int. Cl. A61B 5/16; A61B 5/00; A61B 5/11; A61B 5/103; A61B 5/024

(52) U.S. Cl. CPC A61B 5/163; A61B 5/165; A61B 5/7267; A61B 2560/02; A61B 5/1032; A61B 5/02405; A61B 5/1128

(57) ABSTRACT

A system comprising: at least one hardware processor; and
a non-transitory computer-readable storage medium having stored thereon program instructions, the program instructions executable by the at least one hardware processor to: receive, as input, a video image stream of a bodily region of a subject, continuously extract from said video image stream at least some of: (i) facial parameters of said subject, (ii) skin-related features of said subject, and (iii) physiological parameters of said subject, and apply a first trained machine learning classifier selected from a group of trained machine learning classifiers, based, at least in part, on a detected combination of said facial parameters, skin-related features, and physiological parameters, to determine one or more states of stress in said subject.

### CROSS REFERENCE TO RELATED APPLICATIONS

This application claims priority from Israeli Patent Application No. 262116, filed on Oct. 3, 2018, entitled “REMOTE PREDICTION OF HUMAN NEUROPSYCHOLOGICAL STATE,” the contents of which are incorporated by reference herein in their entirety.

### BACKGROUND

The invention relates to the field of machine learning.

Human psychophysiological behavior can be described as a combination of different physiological stress types. Stress, in turn, may be described as a physiological response to internal or external stimulation, and can be observed in physiological indicators. When external or internal stimulations are created, they may cause the activation of the hypothalamus brain system to activate different processes, which influence the autonomic nervous system and sympathetic and parasympathetic systems, which ultimately control the physiological systems of the human body. Accordingly, measuring physiological responses may serve as an indirect indicator of underlying stress factors in humans subjects.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in an embodiment, a system comprising at least one hardware processor; and a non-transitory computer-readable storage medium having stored thereon program instructions, the program instructions executable by the at least one hardware processor to: receive, as input, a video image stream of a bodily region of a subject, continuously extract from said video image stream at least some of: (i) facial parameters of said subject, (ii) skin-related features of said subject, and (iii) physiological parameters of said subject, and apply a first trained machine learning classifier selected from a group of trained machine learning classifiers, based, at least in part, on a detected combination of said facial parameters, skin-related features, and physiological parameters, to determine one or more states of stress in said subject.

There is also provided, in an embodiment, a method comprising receiving, as input, a video image stream of a bodily region of a subject; continuously extracting from said video image stream at least some of: (i) facial parameters of said subject, (ii) skin-related features of said subject, and (iii) physiological parameters of said subject; and applying a first trained machine learning classifier selected from a group of trained machine learning classifiers, based, at least in part, on a detected combination of said facial parameters, skin-related features, and physiological parameters, to determine one or more states of stress in said subject.

There is further provided, in an embodiment, a computer program product comprising a non-transitory computer-readable storage medium having program instructions embodied therewith, the program instructions executable by at least one hardware processor to: receive, as input, a video image stream of a bodily region of a subject; continuously extract from said video image stream at least some of: (i) facial parameters of said subject, (ii) skin-related features of said subject, and (iii) physiological parameters of said subject; and apply a first trained machine learning classifier selected from a group of trained machine learning classifiers, based, at least in part, on a detected combination of said facial parameters, skin-related features, and physiological parameters, to determine one or more states of stress in said subject.

In some embodiments, said bodily region is selected from the group consisting of whole body, facial region, and one or more skin regions.

In some embodiments, said group of trained machine learning classifiers comprises a hierarchical cascade of machine learning classifiers.

In some embodiments, said applying further comprises selecting a next machine learning classifier for application, from said group of trained machine learning classifiers, based, at least in part, on detecting time-dependent changes in said detected combination of said facial parameters, skin-related features, and physiological parameters.

In some embodiments, said applying comprises selecting a number of machine learning classifiers from said group, and wherein said determining is based, at least in part, on a combination of determinations by each of said classifiers.

In some embodiments, at least one of said machine learning classifiers in said group of trained machine learning classifiers is trained on a training set comprising only one of physiological parameters, skin-related features, and physiological parameters.

In some embodiments, at least one of said machine learning classifiers in said group of trained machine learning classifiers is trained on a training set comprising a combination of two or more of physiological parameters, skin-related features, and physiological parameters.

In some embodiments, each of said training sets further comprises labels associated with one of said states of stress.

In some embodiments, each of said training sets in labelled with said labels.

In some embodiments, said states of stress are selected from the group consisting of neutral stress, cognitive stress, positive emotional stress, and negative emotional stress.

In some embodiments, said determining further comprise detecting a state of global stress in said subject, based, at least in part, on said determined one or more states of stress in said subject.

In some embodiments, said plurality of physiological parameters comprise at least some of a photoplethysmogram (PPG) signal, heartbeat rate, heartbeat variability (HRV), respiration rate, and respiration variability.

In some embodiments, said plurality of skin-related features represent time-dependent spectral reflectance intensity from a skin region of said subject.

In some embodiments, said skin-related features are based, at least in part, on image data values in said video image stream, in at least one color representation model selected from the group consisting of: RGB (red-green-blue), HSL (hue, saturation, lightness), HSV (hue, saturation, value), and YCbCr.

In some embodiments, said plurality of facial parameters comprise at least some of: eye blinking patterns, eye movement patterns, and pupil movement patterns.

In some embodiments, said eye blinking patterns comprise at least some of: changes in eye aspect ratio, duration between successive eyelid closures, duration of eye closure, duration of eye opening, eye blinking rate, and eye blinking rate variability.

In some embodiments, said pupil movements comprise at least some of pupil coordinates change, pupil movement along X-Y axes, acceleration of pupil movement along X-Y axes, and pupil movement relative to eye center.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 is a block diagram of an exemplary system for automated remote analysis of variability in a neurophysiological state in a human subject, according to an embodiment;

FIG. 2 is a block diagram illustrating the functional steps of data acquisition and training set construction, according to an embodiment;

FIG. 3 is a block diagram schematically illustrating an exemplary psycho-physiological test protocol configured for inducing various categories of stress in a subject, according to an embodiment;

FIG. 4 is a block diagram illustrating an exemplary video processing flow, according to an embodiment;

FIG. 5A illustrates the two main ROI detection methods which may be employed by the present invention, according to an embodiment;

FIG. 5B schematically illustrates the processing flow of a video qualification and data recovery methods, according to an embodiment;

FIG. 6A schematically illustrates a process for skin-dependent ROI detection, according to an embodiment;

FIG. 6B illustrates an example of human skin behavior over time;

FIG. 7A schematically illustrates a process for feature extraction based on face-dependent ROI detection, according to an embodiment;

FIG. 7B schematically illustrates a process for eye blinking detection, according to an embodiment;

FIG. 8A schematically illustrates a process for features extraction based on skin-dependent ROI detection, according to an embodiment;

FIG. 8B schematically illustrates a process for the detection of a PPG signal in skin ROI, according to an embodiment;

FIG. 9 schematically illustrates a method for tracking of a biological object in a video image stream, based on skin classification, according to an embodiment;

FIG. 10A schematically illustrates a model switching method, according to an embodiment; and

FIG. 10B is a schematic illustration of a multi-model switching scheme, according to an embodiment.

### DETAILED DESCRIPTION

Disclosed herein are a method, system, and computer program product for automated remote analysis of variability in neurophysiological states in a human subject. In some embodiments, the analysis of neurophysiological states is based, at least in part, on remotely estimating a plurality of physiological, skin-related, muscle movement, and/or related parameters in a subject. In some embodiments, estimating these plurality of parameters may be based on analyzing a video image stream of a head and/or facial region of the subject. In some embodiments, the image stream may include other and/or additional parts of the subject's body, and/or a whole body video image stream.

In some embodiments, an analysis of these remotely-estimated parameters may lead to the detection of psychophysiological and neurophysiological data about the subject. In some embodiments, such data may be correlated with one or more stress states, which may include, but are not limited to:
Neutral stress: A neutral state in which reflects reduced levels of cognitive and/or emotional stress.
Cognitive stress: Stress associated with cognitive processes, e.g., when a subject is asked to perform a cognitive task, such as to solve a mathematical problem.
Positive emotional stress: Stress associated with positive emotional responses, e.g., when a subject is exposed to images inducing positive feelings, such as happiness, exhilaration, delight, etc.
Negative emotional stress: Stress associated with negative emotional responses, e.g., when a subject is exposed to images inducing fear, anxiety, distress, anger, etc.
Continuous expectation stress: A state of suspenseful anticipation, e.g., when a subject is expecting an imminent significant or consequential event.

In some embodiments, the present invention may be configured for detecting a state of ‘global stress’ in a human subject based, at least in part, on detecting a combination of one or more of the constituent stress categories. In some embodiments, a ‘global stress’ signal may be defined as an aggregate value of one or more individual constituent stress states in a subject. For example, a global stress value in a subject may be determined by summing the values of detected cognitive and/or emotional stress in the subject. In some variations, the aggregating may be based on a specified ratio between the individual stress categories.

In some embodiments, the detection of one or more stress states, and/or of a global stress state, may further lead to determining a neurophysiological state associated with a ‘significant response’ (SR) in the subject, which may be defined as consistent, significant, and timely physiological responses in a subject, in connection with responding to a relevant trigger (such as a question, an image, etc.). In some embodiments, detecting an SR state in a subject may indicate an intention on part of the subject to provide a false or deceptive answer to the relevant test question.

In some embodiments, the present invention may be configured for training a machine learning classifier to detect the one or more stress states and/or an SR state in a subject. In some embodiments, a machine learning classifier of the present invention may comprise a group of cooperating, hierarchical classification sub-models, wherein each sub-model within the group may be trained on a different training set associated with specific subsets and/or modalities of physiological features, skin-related, muscle movement parameters, and/or related parameters. In some embodiments, in an inference stage, the group of classification sub-models may be applied selectively and/or hierarchically to an input dataset, depending on, e.g., the types, content, measurement duration, and/or measurement quality of physiological and other parameters available in the dataset.

In some embodiments, the present system may be configured for estimating the physiological and other parameters of a single subject, in a controlled environment. In some embodiments, the present system may be configured for estimating the physiological and other parameters of a single subject while in movement and/or in an unconstrained manner. In some embodiments, the present system may be configured for estimating the physiological and other parameters of one or more subjects in a crowd, e.g., at an airport, a sports venue, or on the street.

A potential advantage of the present invention is, therefore, in that it provides for an automated, remote, quick, and efficient estimation of a neurophysiological state of a subject, using common and inexpensive video acquisition means. In single-subject applications, the present invention may be advantageous for, e.g., interrogations or interviews, to detect stress, SR states, and/or deceitful responses. In crowd-based applications, the present invention may provide for an automated, remote, and quick estimation of moods, emotions, and/or intentions of individuals in the context of large gatherings and popular events. Thus, the present invention may provide for enhanced security and thwarting of potential threats in such situations.

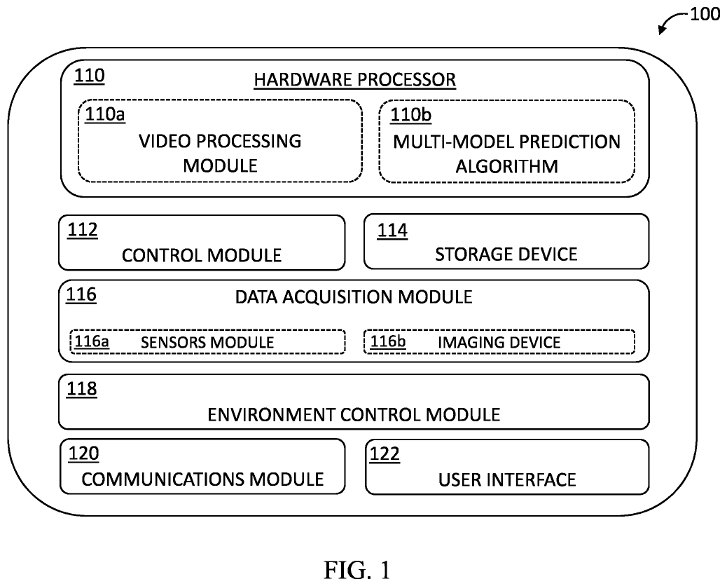

FIG. 1 is a block diagram of an exemplary system 100 according to an embodiment of the present invention. System 100 as described herein is only an exemplary embodiment of the present invention, and in practice may have more or fewer components than shown, may combine two or more of the components, or a may have a different configuration or arrangement of the components. The various components of system 100 may be implemented in hardware, software or a combination of both hardware and software. In various embodiments, system 100 may comprise a dedicated hardware device, or may form an addition to or extension of an existing device.

In some embodiments, system 100 may comprise a hardware processor 110 having a video processing module 110a and a multi-model prediction algorithm 110b; a control module 112; a non-volatile memory storage device 114; a physiological parameters module 116 having, e.g., a sensors module 116a and an imaging device 116b; environment control module 118; communications module 120; and user interface 122.

System 100 may store in storage device 114 software instructions or components configured to operate a processing unit (also “hardware processor,” “CPU,” “GPU,” or simply “processor”), such as hardware processor 110. In some embodiments, the software components may include an operating system, including various software components and/or drivers for controlling and managing general system tasks (e.g., memory management, storage device control, power management, etc.) and facilitating communication between various hardware and software components.

In some embodiments, imaging device 116b may comprise any one or more devices that capture a stream of images and represent them as data. Imaging device 116b may be optic-based, but may also include depth sensors, radio frequency imaging, ultrasound imaging, infrared imaging, and the like. In some embodiments, imaging device 116b may be a Kinect or a similar motion sensing device, capable of, e.g., IR imaging. In some embodiments, imaging device 116b may be configured to detect RGB (red-green-blue) spectral data. In other embodiments, imaging device 116b may be configured to detect at least one of monochrome, ultraviolet (UV), near infrared (NIR), and short-wave infrared (SWIR) spectral data.

In some embodiments, physiological parameters module 116 may be configured for directly acquiring a plurality of physiological parameters data from human subjects, using one or more suitable sensors and similar measurement devices. In some embodiments, sensors module 116a may comprise at least some of:
An infrared (IR) sensor for measuring bodily temperature emissions;
a skin surface temperature sensor;
a skin conductance sensor, e.g., a galvanic skin response (GSR) sensor;
a respiration sensor;
a peripheral capillary oxygen saturation (SpO2) sensor;
an electrocardiograph (ECG) sensor;
a blood volume pulse (BVP) sensor, also known as photoplethysmography (PPG);
a heart rate sensor;
a surface electromyography (EMG) sensor;
an electroencephalograph (EEG) acquisition sensor;
a bend sensor, to be placed on fingers and wrists to monitor joint motion; and/or
sensors for detecting muscle activity in various areas of the body.

In some embodiments, environment control module 118 comprises a plurality of sensors and measurement devices configured for monitoring environmental conditions at a testing site. Such sensors may include, e.g., lighting and temperature conditions, to ensure consistency in environmental conditions among multiple test subjects. For example, environment control module 118 may be configured for monitoring an optimal ambient lighting in the test environment between 1500-3000 lux units, e.g., 2500. In some embodiments, environment control module 118 may be configured to monitor an optimal ambient temperature in the test environment, e.g., between 22-24° C.

In some embodiments, communications module 120 may be configured for connecting system 100 to a network, such as the Internet, a local area network, a wide area network and/or a wireless network. Communications module 120 facilitates communications with other devices over one or more external ports, and also includes various software components for handling data received by system 100. In some embodiments, a user interface 122 comprises one or more of a control panel for controlling system 100, display monitor, and a speaker for providing audio feedback. In some embodiments, system 100 includes one or more user input control devices, such as a physical or virtual joystick, mouse, and/or click wheel. In other variations, system 100 comprises one or more of a peripherals interface, RF circuitry, audio circuitry, a microphone, an input/output (I/O) subsystem, other input or control devices, optical or other sensors, and an external port. Each of the above identified modules and applications correspond to a set of instructions for performing one or more functions described above. These modules (i.e., sets of instructions) need not be implemented as separate software programs, procedures or modules, and thus various subsets of these modules may be combined or otherwise re-arranged in various embodiments. In some embodiments, control module 112 is configured for integrating, centralizing and synchronizing control of the various modules of system 100.

An overview of the functional steps in a process for automated remote analysis of a neurophysiological state in a human subject, using a system such as system 100, will be provided within the following sub-sections.

### Training a Multi-Model Prediction Algorithm

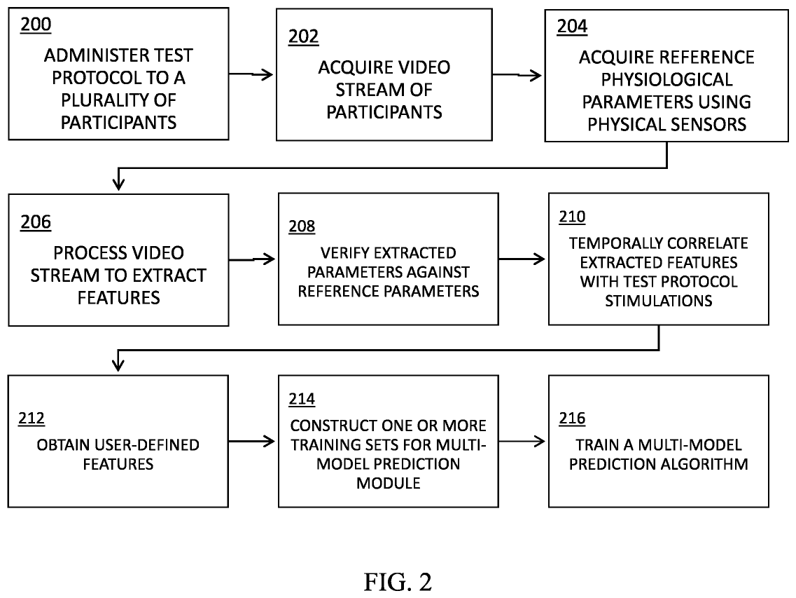

FIG. 2 is a block diagram illustrating the functional steps of data acquisition and training set construction, according to some embodiments.

As noted above, the present invention may be configured for remotely estimating a plurality of physiological, skin-related, muscle movement, and/or related parameters. In some embodiments, these parameters may be used for extracting a plurality of features including, but not limited to:
A plurality of facial-related parameters, including, but not limited to, face orientation, face geometry, eye blinking patterns, and/or pupil movement;
a plurality of skin-related features associated with spectral reflectance intensity and/or light absorption of a skin region; and
a plurality of physiological parameters which may include, e.g., a photoplethysmogram (PPG) signal, a heart rate, heart rate variability (HRV), respiratory rate, and/or derivatives thereof.

As shall be further explained below under “Inference Stage—Applying the Multi-Model Prediction Algorithm,” in real life subject observation situations, several challenges emerge related to subject movement, lighting conditions, system latency, facial detection algorithms limitations, the quality of the obtained video, etc. For example, observed subjects may not remain in a static posture for the duration of the observation, so that, e.g., the facial region may not be fully visible at least some of the time. In another example, certain features may suffer from time lags due to system latency. For example, HRV frequency domain features may take in some instances between 40 seconds and 5 minutes to come online.

Accordingly, the predictive model of the present invention may be configured for adapting to a variety of situations and input variables, by switching among a number of predictive sub-models configured for various partial-data situations. In some embodiments, multi-model prediction algorithm 110b may thus be configured for providing continuous uninterrupted real-time analytics in situations where not all features are extractable from the data stream because, e.g., a facial region is not visible in the video stream, or in periods of data latency when not all features have come online yet. For example, multi-model prediction algorithm 110b may be configured for switching between, e.g., two sets of predictive models (e.g., one for both facial region and skin features, and the other for skin features only), depending on facial region detectability in the video stream. In addition, within each of the sets, different sub-models may be configured for classification based on different combinations of features in their respective modalities.

Accordingly, a training set for multi-model prediction algorithm 110b may comprise a plurality of training sub-sets, each configured for training within a different modality and/or a different partial-features situation.

In some embodiments, at a training stage, system 100 may be configured for acquiring one or more datasets for use in generating the plurality of training sets for multi-model prediction algorithm 110b. In some embodiments, the training sets may be configured for reflecting physiological characteristics changes in a plurality of human subjects associated with the various states of stress noted above (i.e., neutral stress, cognitive stress, emotional negative stress, emotional positive stress, and expectation stress). In some embodiments, the trainings sets may be configured for isolating, in each human subject, the characteristics and physiological changes associated with each stress type, so as to determine the types of physiological mechanisms that are activated or inactivated during each stress state (e.g., sympathetic and para-sympathetic systems) and their corresponding reaction times.

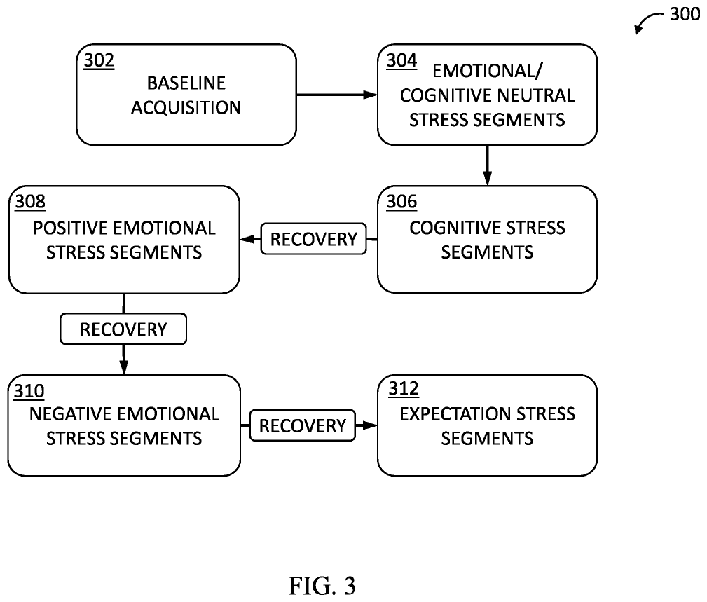

In some embodiments, a dataset for generating training sets for the present invention may comprise acquiring a plurality of muscle movement, skin-related, physiological, and related parameters from human test subjects, wherein the parameters are being acquired in the course of administering one or more psycho-physiological test protocols to each of the subjects (as will be further described below with reference to FIG. 3). In some embodiments, a data set generated by system 100 for the purpose of generating the training set may be based on physiological parameters data acquired from between 30 and 450 test subjects, e.g., 150 test subjects. In other embodiments, the number of subjects may be smaller or greater. In some embodiments, all subjects may undergo identical test protocols. In other embodiments, sub-groups of test subjects selected at random from a pool of potential subjects may be administered different versions of the test protocol.

With continued reference to FIG. 2, in some embodiments, at a step 200, a test protocol may be administered by a specialist, be a computer-based test, or combine both approaches. in cases where a test protocol may be administered by a specialist, test subjects may be seated near the specialist so as to induce a degree of psychological pressure in the subject, however, in such a way that test subject and specialist do not directly face each other, to avoid any undue influence of the specialist on the subject. In addition, subjects may be instructed to sit upright, with both legs touching the ground, and to avoid, to the extent possible, body, head, and/or hand movements.

In some embodiments, test subjects may be selected from a pool of potential subjects comprising substantially similar numbers of adult men and women. In some embodiments, potential test subjects may undergo a health and psychological screening, e.g., using a suitable questionnaire, to ensure that no test subject has a medical and/or mental condition which may prevent the subject from participating in the test, adversely affect test results, and/or manifest in adverse side effects for the subject. For example, test subjects may be screened to ensure to no test subject takes medications which may affect test results, and/or currently or generally suffers adverse health conditions, such as cardiac disease, high blood pressure, epilepsy, mental health issues, consumption of alcohol and/or drugs within the most recent 24 hours, and the like.

In some embodiments, at a step 202, imaging device 116b may be configured for continuously acquiring, during the course of administering the test protocol to each subject, a video image stream of the whole body, the facial region, the head region, one or more skin regions, and/or other body parts, of the subject.

In some embodiments, at a step 204, data acquisition module 116 may be configured for, simultaneously acquiring a plurality of reference physiological parameters from the subject. In some embodiments, such reference physiological parameters may be used to verify one or more of the features extracted from the video stream. For example, sensors module 116a may be configured for taking measurements relating to bodily temperature; heart rate; heart rate variation (HRV); blood pressure; blood oxygen saturation; skin conductance; respiratory rate; eye blinks; ECG; EMG; EEG; PPG; finger/wrist bending; and/or muscle activity. Similarly, environment control module 118 may be configured for continuously monitoring ambient conditions during the course of administering the test protocol, including, but not limited to, ambient temperature and lighting.

In some embodiments, each psycho-physiological test protocol may comprise a series of between 2 and 6 stages. During each of the stages, subjects may be exposed to between 1 and 4 stimulation segments, each configured to induce one of the different categories of stress described above, including neutral emotional or cognitive stress, cognitive stress, positive emotional stress, negative emotional stress, and/or continuous expectation stress. In some embodiments, each test stage may last between 20 and 600 seconds. In some embodiments, all stages have an identical length, e.g., 360 seconds. In some embodiments, each segment within a stage may have a length of between 10 and 400 seconds. In some embodiments, test segments designed to induce continuous expectation stress may be configured for lasting at least 360 seconds, so permit the buildup of suspenseful anticipation.

In some embodiments, the various stages and/or individual segments within a stage may be interspersed with periods of break or recovery configured for unwinding a stress state induced by the previous stimulation. In some embodiments, each recovery segment may last, e.g., 120 seconds. In some embodiments, recovery segments may comprise exposing a subject to, e.g., relaxing or meditative background music, changing and/or floating geometric images, and/or simple non-taxing cognitive tasks. For example, because emotional stress stimulations may have a heightened and/or more lasting effect on subjects, recovery segments following negative emotional stimulations may comprise simple cognitive tasks, such as a dots counting task, configured for neutralizing an emotional stress state in a subject.

FIG. 3 is a block diagram schematically illustrating an exemplary psycho-physiological test protocol 300 configured for inducing various categories of stress in a subject, according to an embodiment. In some embodiments, at a stage 302, system 100 may be configured for acquiring baseline physiological parameters of a test subject, in a state of rest where the subject may not be exposed to any stimulations.

At a stage 304, the subject may be exposed to one or more stimulations configured to induce a neutral emotional or cognitive state. For example, the subject may be exposed to one or more segments of relaxing or meditative background music, to induce a neutral emotional state. The subject may also be exposed to images incorporating, e.g., changing geometric or other shapes, to induce a neutral cognitive state.

Following the neutral stress stage, at a stage 306, the subject may be exposed to one or more cognitive stress segments, which may be interspersed with one or more recovery segments. For example, the subject may be exposed to a Stroop test asking the subject to name a font color of a printed word, where the word meaning and font color may or may not be incongruent (e.g., the word ‘Green’ may be written variously using a green or red font color). In other cases, a cognitive stimulation may comprise a mathematical problem task, a reading comprehension task, a ‘spot the difference’ image analysis task, a memory recollection task, and/or an anagram or letter-rearrangement task. In some cases, each cognitive task may be followed by a suitable recovery segment.

At a stage 308, the subject may then be exposed to one or more stimulation segments configured to induce a positive emotional response. For example, the subject may be exposed to one or more video segments designed to induce reactions of laughter, joy, happiness, and the like. Each positive emotional segment may be followed by a suitable recovery segment.

At a stage 310, the subject may be exposed to one or more stimulations configured to induce a negative emotional response. For example, the subject may be exposed to one or more video segments designed to induce reactions of fear, anger, distress, anxiety, and the like. Each negative emotional segment may be followed by a suitable recovery segment.

Finally, at a stage 312, the subject may be exposed to one or more stimulations configured to induce continuous expectation stress. For example, the subject may be exposed to one or more video segments showing a suspenseful scene from a thriller feature film. Each expectation segments may be also followed by a suitable recovery segments.

Exemplary test protocol 300 is only one possible such protocol. Alternative test protocols may include fewer or more stages, may arrange the stages in a different order, and/or may comprise a different number of stimulation and recovery segments in each stage. However, in some embodiments, test protocols of the present invention may be configured to place, e.g., a negative emotional segment after a positive emotional segment, because negative emotions may be lingering emotions which may affect subsequent segments.

With reference back to FIG. 2, at a step 206, following the acquisition of the video stream from a predetermined number of test subjects using, e.g., test protocol 300, video processing module 110a may be configured for processing the video stream of each subject using the methods described below under “Video Processing Methods—ROI Detection” and “Video Processing Methods—Feature Extraction,” to extract a plurality of features.

At 208, at least some of the extracted features may be verified against the reference data acquired in step 204, to validate the video processing methods disclosed herein. At 210, video processing module 110a may be configured for labelling the training datasets, e.g., by temporally associating the extracted features for each test subject with the corresponding stimulation segments administered to the subject, using appropriate time stamps. In some embodiments, such labelling may be supplemented with manual labeling of the features by, e.g., a human specialist.

At 212, system 100 may be configured for obtaining a plurality of user-generated input data points, e.g., through user interface 122. Stress prediction models are based on many physiological data which can be dependent, e.g., on age, gender, and/or skin tones. For example, various skin tones may generate different levels of artifacts in remotely-obtained PPG signal. Accordingly, in some embodiments, system 100 may be configured for obtaining and taking into account a plurality of user-defined features, such as:
Age (e.g., an age range: 18-25, 25-35, 35-45, 45-55, etc.);
gender; and/or
skin tone (e.g., defined as a color range in RGB values or based on the Fitzpatrick skin typing scale).

At 214, the temporally-associated dataset may be used to construct one or more labeled training sets for training one or more models of multi-model prediction algorithm, 110b to predict one or more of the constituent stress categories (i.e., neutral stress, cognitive stress, positive emotional stress, negative emotional stress, and/or expectation stress). In some embodiments, each training set may include a different combination of one or more features configured for training an associated sub-model to predict states of stress based on that specified combination of features.

Finally, at a step 216, the training sets generated using the process described above are used to train the multi-model prediction algorithm described below under “Inference Stage—Applying the Multi-Model Prediction Algorithm.”

### Video Processing Methods—ROI Detection

In some embodiments, the present invention provides for the processing of an acquired video stream by video processing module 110a, to extract a plurality of relevant features. In some embodiments, video processing module 110a may be configured for detecting regions-of-interest (ROI) in the video stream which comprise at least one of:
A facial region of the subject, from which such features as facial geometry, facial muscles activity, facial movements, and/or eye-related activity, may be extracted; and
skin regions, from which one or more physiological parameters may be extracted.

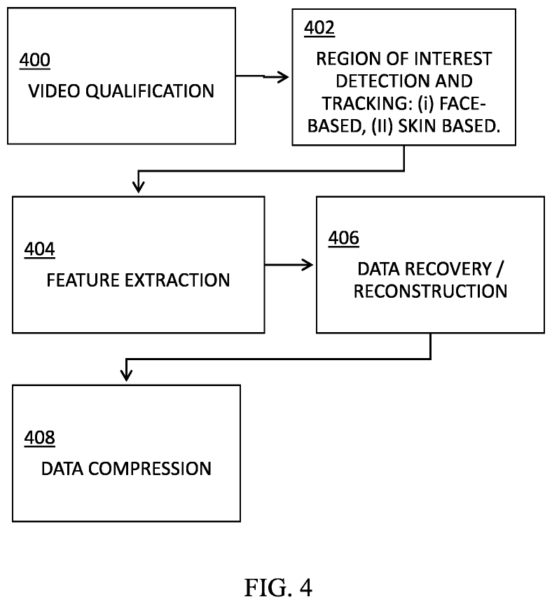

FIG. 4 is a block diagram illustrating an exemplary video processing flow, according to an embodiment.

In some embodiments, at a step 400, video processing module 110a may be configured for performing a qualification stage of the video stream. For example, video qualification may comprise extracting individual image frames to determine, e.g., subject face visibility, face size relatively to frame size, face movement speed, image noise level, and/or image luminance level. Some or all of these parameters may be designated as artifacts and output as a times series, which may be temporally-correlated with the main video processing time series. The artifacts time series may then be used for estimating potential artifacts in the video stream, which then potentially may be used for data recovery in sections where artifacts make the data series too noisy, as shall further be explained below.

In some embodiments, at a step 402, video processing module 110a may be configured for performing region-of-interest (ROI) detection to detect a facial region, a head region, and/or other bodily regions of each subject.

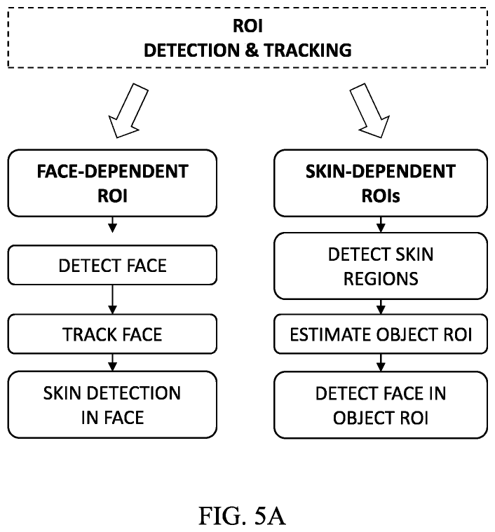

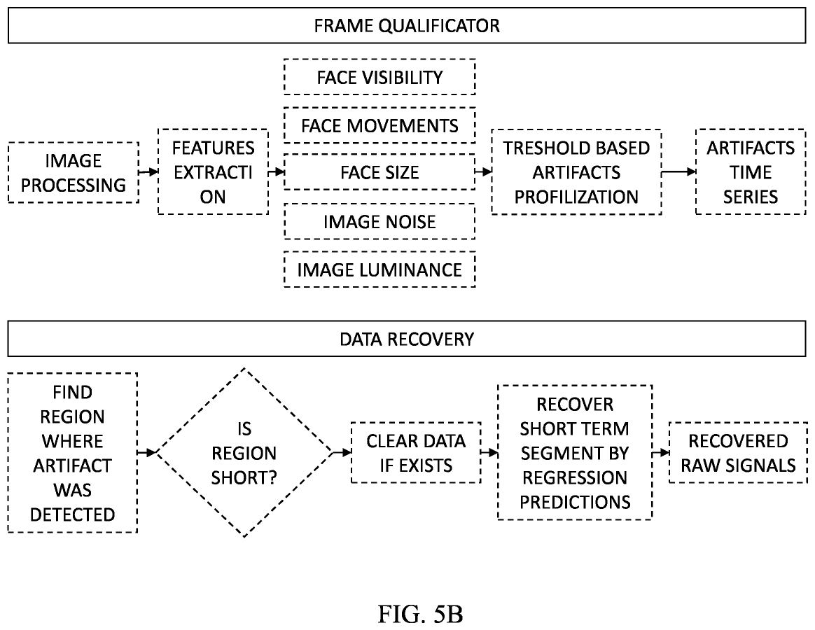

FIG. 5A illustrates the two main ROI detection methods which may be employed by the present invention:
Face-dependent ROI detection, and
Skin-dependent ROI detection.

I. Face-Dependent ROI Detection

This method relies on detecting and tracking a facial region in the video image stream, based, at least in part, of a specified number of facial features and landmarks. Once a facial region has been identified, video processing module 110a may then be configured for tracking the facial region in the image stream, and for further identifying regions of skin within the facial region (i.e., those regions not including such areas as lips, eyes, hair, etc.).

In some embodiments, to reduce computational demands on system 100 when processing a high-definition video stream, video processing module 110a may be configured for performing facial tracking using the following steps:
Resizing a high resolution video stream, e.g., to a size of 640×480 pixels, while saving the resizing coefficients for possible future coordinates restoration to match the original frame size;
detecting a face in a resized frame, based on one or more known face detection algorithms;
initializing one or more known tracking algorithms to track the detected face rectangle in the image stream;
once the tracking algorithm has found an updated position of the face in a subsequent frame, resizing the updated coordinates to the original coordinates to match the source resolution; and
detecting facial landmark points on the updated facial region and outputting the facial landmark points and facial rectangle position.

In case the facial tracking loses the face in a subsequent frame, video processing module 110a may be further configured for:
Taking the rectangle coordinates of the previously-detected frame;
iteratively expanding the region of the facial rectangle by, e.g., 10-15% at a time, to try to find the face by using one or more known face detection algorithms;
continuing expanding the search region at every iteration until a face is found; and
once a face has been found, continuing to track the face as described above.

In some embodiments, video processing module 110a may further be configured for detecting skin regions within the detected face in the image stream, based, at least in part, on using at least some of the facial landmark points detected by the previous steps for creating a face polygon. This face polygon may then be used as a skin ROI. Because facial regions also contain non-skin parts (such as eyes, lips, and hair), the defined polygon ROI cannot be used as-is. However, because the defined polygon includes mainly skin parts, statistical analysis may be used for excluding the non-skin parts, by, e.g.:
Calculating a mean value and standard deviation of all pixels in each of the red, green and blue (RGB) channels; and
denoting as non-skin pixels all those pixels having a channel value that is smaller than mean−alpha*std or larger than mean+alpha*std.

At a step 406 in FIG. 4, video processing module 110a may be configured for performing data recovery with respect to image stream portions where potential artifacts may be present. FIG. 5B schematically illustrates the processing flow of a video qualification and data recovery methods, according to an embodiment. In some embodiments video processing module 110a may be configured for performing a video qualification stage, wherein all video frames are processed for estimating and extracting a set of one or more factors which can point to the existence of potential artifacts and/or the overall quality of the stream. In some embodiments, the extracted factors may include, e.g., face visibility, face size relatively to frame size, face movement speed, image noise level, and/or image luminance level. In some embodiments, the qualification stage is performed simultaneously with the main video processing flow described in this section. In some embodiments, video processing module 110a may be configured for outputting an artifacts time series which may be temporally correlated with the video stream.

In some embodiments, to recover video stream regions affected by artifacts, video processing module 110a may be configured for applying a sliding window of, e.g., 10 seconds, to the stream, to identify regions of at least 5 seconds of continuously detected artifacts, based on the time series determined in the qualification stage. For each such 5 seconds region, video processing module 110a may be configured for using regression prediction for predicting the a 10-seconds window data, based, at least in part, on the previous samples in the time series.

II. Skin-Dependent ROI Detection:

This method begins with detecting skin regions in the image stream (as noted, these are regions not including such areas as lips, eyes, hair, etc.). Based on skin detection, video processing module 110a may then be configured for detecting a facial region in the skin segments collection.

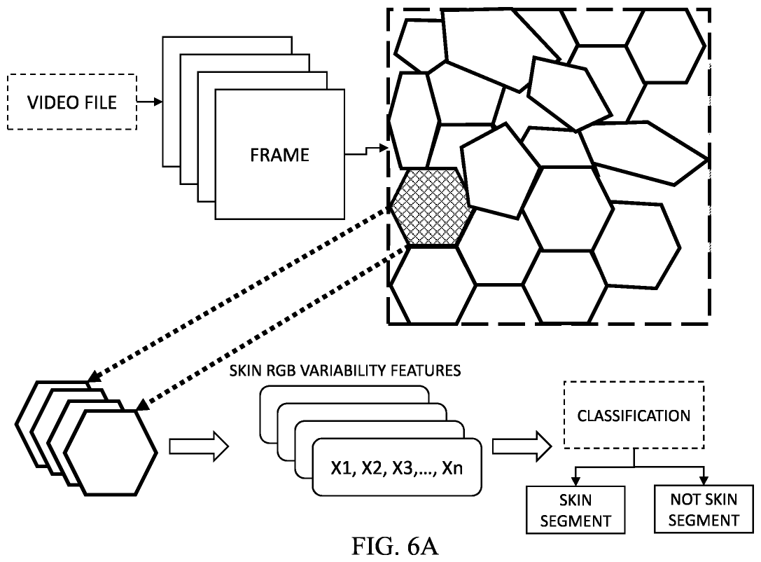

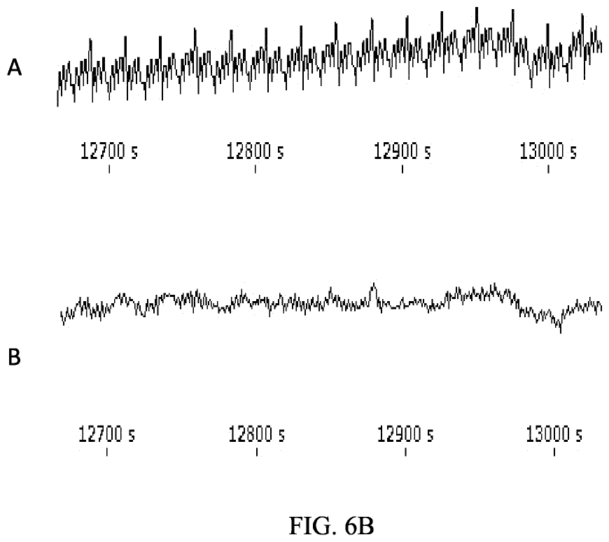

FIG. 6A schematically illustrates a process for skin-dependent ROI detection, according to an embodiment. In some embodiments, video processing module 110a may be configured for receiving and segmenting a video image frame into a plurality of segments, and then performing the following steps:
Defining a polygon for each segment and initializing a tracking of polygon points in subsequent frames;
for each new position of every segment in a subsequent frame, calculating mean values of pixels in the segment, e.g., for each RGB channel;
adding these calculated pixel values features into overlapping window of between 2-5 seconds; and
applying, e.g., a machine learning classifier to the window, to determine whether a time-series of each RGB channel in the segment may be classified as human skin behavior, based, at least in part, on specified human biological patterns, such as
   typical human skin RGB color ranges, and
   typical human skin RGB color variability over time (which may be related to such parameters as blood oxygenation).

FIG. 6B illustrates an example of light absorption and spectral reflectance associated with human skin. For example, the metrics of spectral reflectance received from objects are dependent, at least in part, on the optical properties of the captured objects. Hence, the spectral reflectance received from live skin is dependent on the optical properties of the live skin, with particular regard to properties related to light absorption and scattering. When a light beam having a specific intensity and wavelength is radiated at a live skin irradiation point, part of this light beam is diffusely reflected from the surface of the skin, while another part of the light beam passes through the surface into the tissue of the skin, and distributes there by means of multiple scattering. A fraction of this light scattered in the skin exits back out from the skin surface as visible scattered light, whereby the intensity of this scattered light depends on the distance of the exit point from the irradiation point as well as on the wavelength of the light radiated in. This dependence is caused by the optical material properties of the skin. For example, different spectral bands (with different wavelengths) of the spectrum have different absorption levels in the live skin. Thus, green light penetrates deeper than red or blue light, and therefore the absorption levels, and hence reflectance, of the red and the blue bands are different. Thus, different absorption levels of different wavelengths can lead to different metrics of spectral reflectance. Accordingly, these unique optical properties may be used for detection and tracking purposes.

Panel A in FIG. 6B illustrates the behavior of non-skin material, where the signal (showing blue channel values) reflects light such that a source's blinking frequency may be indicated by the graph. In contrast, human skin (Panel B) does not reflect the light as efficiently, so source frequency cannot be discerned from the graph.

In some embodiments, when it is determined that a segment of the segments should be classified as a skin segment, it is added to an array structure. When all skin segments are collected, a bounding rectangle of all skin-segments in the image stream may be estimated. In some embodiments, video processing module 110a may then be configured for detecting facial coordinates and landmarks within the bounding rectangle, which may lead to detecting a facial region.

### Video Processing Methods—Feature Extraction

With reference back to FIG. 4, in some embodiments, at a step 404, video processing module 110a may be configured for extracting:
A plurality of facial-related parameters from the image stream, including, but not limited to, face geometry, eye blinking patterns, and/or pupil movement;
a plurality of skin-related features associated with spectral reflectance intensity of a skin region; and
a plurality of physiological parameters which may include, e.g., a photoplethysmogram (PPG) signal, a heart rate, heart rate variability (HRV), respiratory rate, and/or derivatives thereof.

I. Facial Features

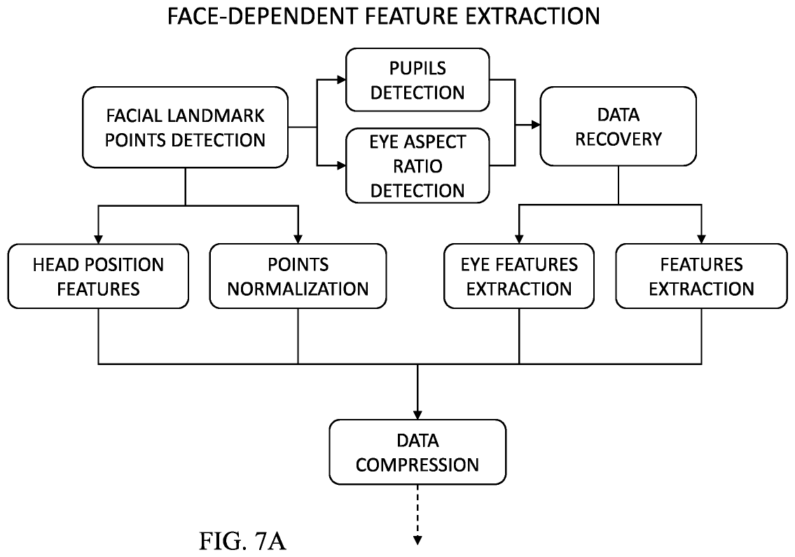

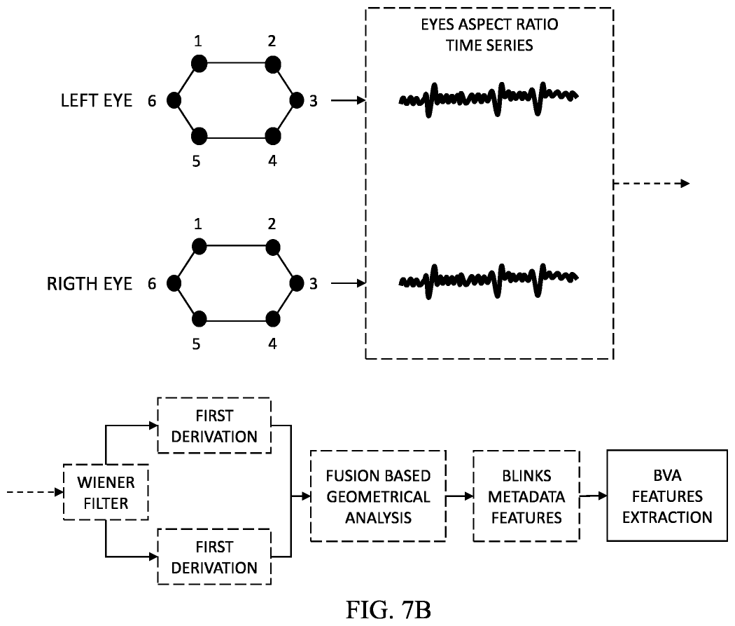

FIG. 7A schematically illustrates a process for feature extraction based on face-dependent ROI detection, according to an embodiment. In some embodiments, following face-dependent ROI detection, facial landmark detection, and, optionally, data recovery, video processing module 110a may be configured for extracting a plurality of facial-related parameters from the image stream, including, but not limited to, face geometry, eye blinking patterns, and/or pupil movement.

In some embodiments, facial geometry detection is based on a plurality of facial landmarks (e.g., 68 landmarks) which allow the extraction of statistical parameters which describe, e.g., face muscle activity as well as face/head movement along X-Y axes. In some embodiments, these parameters are represented as vectors which describe the changes in length and degrees between the facial points over time. In other embodiments, fewer or more facial landmarks, and/or fewer or more parameters may be incorporated into the face geometry analysis.

FIG. 7B schematically illustrates a process for eye blinking detection, according to an embodiment. In some embodiments, extraction of eye blinking features is based, at least in part, on estimating the eye aspect ratio signal which can be constructed by using eye geometrical points from detected polygons and facial landmarks, as described above. The challenge to estimating and analyzing eye blinking variability lies in the fact that eye blinking can be detected only after the blink has occurred. Accordingly, in some embodiments, a sliding window may be used for storing a raw aspect ratio time series, which is then analyzed as a whole for detecting the existing blinks within that window. In some embodiments, video processing module 110a may then be configured for applying, e.g., a Wiener filter to remove noise form the sliding window. Video processing module 110a may then be configured for calculating a first derivative for the aspect ratio signal of each eye, wherein both first derivatives are used for extracting a fusion-based geometrical metadata about the subject's blinking. Then, eye blinking variability analysis may be performed, wherein features matrices related to the sliding windows of each of the left and right eyes are derived. The features matrices may then be used for reconstructing the time series for each feature, so as to keep all data synchronized. Table 1 includes exemplary features which may be extracted using the process described above for eye blinking detection:

TABLE 1
Eye Blinking Feature Set

| Feature Name | Description |
|---|---|
| B left ar | Changes in aspect ratio of the left eye, based on 4 points |
| B right ar | Changes in aspect ratio of the left eye, based on 4 points |
| B left dar | Derivation of changes in aspect ratio of the left eye. |
| B right dar | Derivation of changes in aspect ratio of the right eye. |
| Blink | Time duration between the moment when the eyelids |
| Duration | closure begins to the moment that the eyelids opening |
| | ends. |
| Blink Rate | Number of blinks per millisecond. |
| Blink | Time duration between the blinks. |
| to Blink | |
| Interval | |
| Time to Open | The time duration during which the eyes were open. |

In some embodiments, eye blinking detection may be based on pupil movement detection. In such cases, the method described above may be used to extract a pupils features set, from which eye blinking may be derived. Table 2 includes an exemplary pupil movement feature set.

TABLE 2
Pupil Movement Feature Set

| Feature Name | Description |
|---|---|
| p_rightPupil_x, | Coordinates of right pupil. |
| p_rightPupil_y | |
| p_leftPupil_x, | Coordinates of left pupil. |
| p_leftPupil_y | |
| p_moveX_rightEye, | Describes the movement of the right pupil |
| p_moveY_rightEye | along an X and Y axis. |
| p_moveX_leftEye, | Describes the movement of the left pupil |
| p_moveY_leftEye | along an X and Y axis. |
| p_left_right_rightEye | Relative movement of the right pupil to the |
| | midpoint (distance from the center of the eye). |
| p_left_right_leftEye | Relative movement of the left pupil to the |
| | midpoint (distance from the center of the eye). |
| p_accelX_rightEye, | Acceleration of right pupil along an X and Y |
| p_accelY_rightEye | axes (derivation of moveX, moveY). |
| p_accelX_leftEye, | Acceleration of left pupil along an X and Y |
| p_accelY_leftEye | axes (derivation of moveX, moveY). |

II. Skin-Related Features

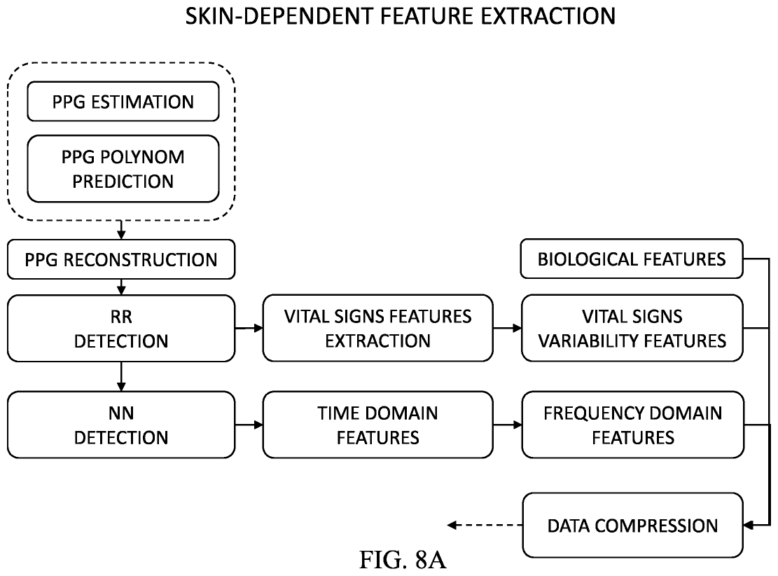

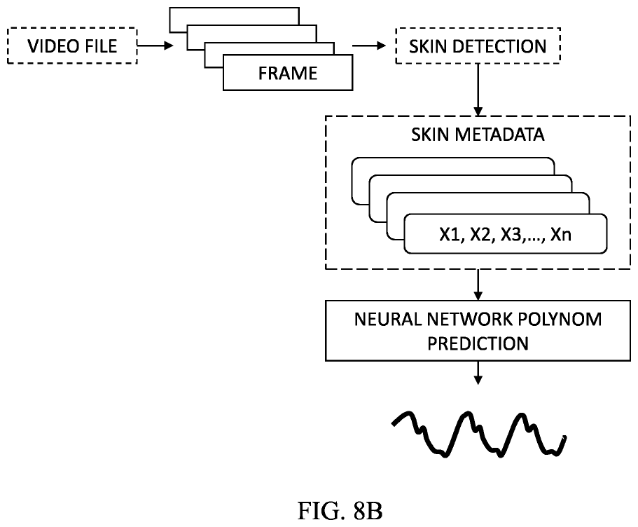

FIG. 8A schematically illustrates a process for features extraction based on skin-dependent ROI detection. In some embodiments, one or more physiological parameters may be extracted from the image stream, including, but not limited to, a PPG signal, heart rate, heart rate variability (HRV), respiratory rate, and/or derivatives thereof.

In some embodiments, the extraction of physiological parameters is based, at least in part, on skin-related features extracted from the images. For example, video processing module 110a may be configured for extracting skin metadata comprising a plurality of skin parameters related, e.g., to color changes within the RGB format. Table 3 includes an exemplary set of such metadata set.

In some embodiments, skin-related feature extraction may be based at least in part, on extracting features from data representing one or more images, or a video stream from an imaging device, e.g., imaging device 116b. In some embodiments, the video stream may be received as an input from an external source, e.g., the video stream can be sent as an input from a storage device designed to manage a digital storage comprising video streams.

In some embodiments, the system may divide the video stream into time windows, e.g., by defining a plurality of video sequences having, e.g., a specified duration, such as a five-second duration. In such an exemplary case, the number of frames may be 126, for cases where the imaging device captures twenty-five (25) frames per second, wherein consecutive video sequences may have a 1-frame overlap. In some embodiments, more than one sequence of frames may be chosen from one video stream. For example, two or more sequences of five seconds each can be chosen in one video stream.

the video processing module 110a may be configured to detect a region-of-interest (ROI) in some or all of the frames in the video sequence, wherein the ROI is potentially associated with live skin. In some embodiments, video processing module 110a may be configured to perform facial detection, a head region, and/or other bodily regions. In some embodiments, an ROI may comprise part of all of a facial region in the video sequence (e.g., with non-skin areas, such as eyes, extracted). In some embodiments, ROI detection may be performed by using any appropriate algorithms and/or methods.

In some embodiments, the detected ROI (e.g., facial skin region) may undergo a segmentation process, e.g., by employing video processing module 110a. In some embodiments, the segmentation process may employ diverse methods for partitioning regions in a frame into multiple segments. In some embodiments, algorithms for partitioning the ROI by a simple linear iterative clustering may be utilized for segmenting the ROI. For example, a technique defining clusters of super-pixels may be utilized for segmenting the ROI. In some embodiments, other techniques and/or methods may be used, e.g., techniques based on permanent segmentation, as further detailed below.

In some embodiments, the segments identified in the first frame of the sequence may also be tracked in subsequent frames throughout the sequence, as further detailed below. In some embodiments, tracking segments throughout a video sequence may be performed by, e.g., checking a center of mass adjustment and polygon shape adjustment between consecutive frames in the sequence. For example, if a current frame has smaller number of segments than a previous frame, missing one or more segments may be added at the same location as in the previous frame.

In some embodiments, an image data processing step may be performed, e.g., by employing video processing module 110a, to derive relevant data with respect to at least some of the segments in the ROI. In some embodiments, the processing stage may comprise data derivation, data cleaning, data normalization, and/or additional similar operations with respect to the data.

In some embodiments, the present disclosure may then provide for determining a set of values for each of the segments in the ROI, for example using an RGB (red-green-blue) color representation model, and/or other or additional models such as HSL (hue, saturation, lightness) and HSV (hue, saturation, value), YCbCr, etc. In some embodiments, the set of values may be derived in a time-dependent manner, along the length of a time window within the video stream. In some embodiments, a variety of statistical and/or similar calculations may be applied to the derived image data values.

In some embodiments, the image data processed may be used for calculating a set of features. In some embodiments, a plurality of features represent time-dependent spectral reflectance intensity, as further detailed below.

In some embodiments, an image data processing stage may comprise at least some of data derivation, data cleaning, data normalization, and/or additional similar operations with respect to the image data.

In some embodiments, for each segment in the ROI in the video sequence, the present algorithm may be configured to calculate an average of the RGB image channels, e.g., in a segment of time windows with a duration of 5 seconds and/or at least 125 frames (at a frame rate of 25 fps) each. in some embodiments, each time window comprises, e.g., 126 frames, wherein the time windows may comprise a moving time window with an overlap of one or more frames between windows.

In some embodiments, utilizing the color channels in the segment involves identifying the average value of each RGB channel in each tracked segment and/or tracked object. In some embodiments, calculating channel values is based on the following derivations:

$$\begin{matrix}{R}_{a v g} ( i ) = \frac{1}{N} {\sum }_{c}^{Col} {\sum }_{r}^{R o w} {R}_{c , r} ( i ) , & ( 1.1 ) \\ {G}_{a v g} ( i ) = \frac{1}{N} {\sum }_{c}^{Col} {\sum }_{r}^{R o w} {G}_{c , r} ( i ) , & ( 1.2 ) \\ {B}_{a v g} ( i ) = \frac{1}{N} {\sum }_{c}^{Col} {\sum }_{r}^{R o w} {B}_{c , r} ( i ) . & ( 1.3 )\end{matrix}$$

In such an exemplary case, r denotes the row and c denotes the column indexes that detect the segment boundaries, N denotes the total number of pixels of the segment corresponding to a specific frame i, and R, G and B denote the number of red, green and blue pixels respectively.

In some embodiments, a preprocessing stage of cleaning the data, e.g., noise reduction for each tracked segment, may be conducted. In one exemplary embodiment, cleaning the data may be processed by, e.g., normalizing the Red, Green, and Blue channels (in RGB Color model), by:

$$[ r ( i ) , g ( i ) , b ( i ) ] = \{ \frac{R ( i )}{R ( i ) + G ( i ) + B ( i )} , \frac{G ( i )}{R ( i ) + G ( i ) + B ( i )} , \frac{B ( i )}{R ( i ) + G ( i ) + B ( i )} \} , \\ i = frame index . \qquad ( 2 )$$

In some embodiments, wherein features may be derived in the frequency domain, data cleaning may comprise, e.g., reducing a DC offset in the data based on a mean amplitude of the signal waveform:

filtered_{DC}=channel−mean(channel), channel=r, g, b. (3)

In some embodiments, the preprocessing stage may further comprise applying, e.g., a bandpass filter and/or another method wherein such filter may be associated with a heart rate of a depicted human. In some embodiments, such bandpass filter has a frequency range of, e.g., 0.75-3.5 Hz, such as an Infinite Impulse Response (IIR) elliptic filter with bandpass ripple of 0.1 dB and stopband attenuation of 60 dB:

signal_band_rgb(c)=filtered_{DC}(c)*BP,c=channel r, g, b (4)

### Spectral Reflectance Intensity Feature Extraction

In some embodiments, a plurality of features can be. In some other embodiments, other calculation methods and formulas may be appreciated by a person having ordinary skills in the art. In some embodiments, the objective of the feature extraction step if to select a set of features which optimally predict live skin in a video sequence

In some embodiments, the plurality of skin-related features selected for representing time-dependent spectral reflectance intensity may comprise at least some of:
Frequency peak for the green channel;
The sum of the area under the curve (AUC) of the 3 RGB channels in the frequency domain;
Sum of the amplitudes of the 3 components, after applying ICA on the RGB channels;
Sum of the AUC in the time domain of the 3 absolute components, after applying ICA on the RGB channels;
Maximum of the AUC in the time domain between the 3

absolute components, after applying ICA on the RGB channels;
Mean of the frequency peak of the 3

components, after applying ICA and Fourier transform on the RGB channels;
Time index of the first peak for the green channel after calculation of an autocorrelation signal;
Frequency peak for the green channel, after calculation of an autocorrelation signal and Fourier transform;
Frequency peak for the hue channel in the HSV model;
AUC of the hue channel in the frequency domain;
Amplitudes of the hue channel in the HSV model in the time domain;
AUC of the absolute hue channel in the HSV model in the time domain;
Time index of the first peak for the hue channel in the HSV model, after calculation of an autocorrelation signal.
Frequency peak for the hue channel in the HSV model after calculation of an autocorrelation signal and Fourier transform;
The number of peaks above a threshold in the hue channel in the HSV model in the time domain;
The highest peak range in the hue channel in the HSV model in the time domain;
The number of rules that exists in the RGB, HSV and YCbCr format.

In some embodiments, additional and/or other features may be used, including:
Channel average: c_{avg}, c=r,g,b:

$$c\_avg = \frac{1}{N} {\sum }_{i = 1}^{N} Channel ( i ) ;$$

Channel standard deviation: c_{std}, c=r,g,b:

$$c\_std = \sqrt{\frac{1}{N - 1} {\sum }_{i = 1}^{N} Channel ( i ) - c\_avg} ;$$

Multiple Channel average: cₙcₘ_{_{avg}}, c_{n/m}=r,g,b. Calculate the feature for the same channel or between different channels:

$${c}_{n} {c}_{m} \_avg = \frac{1}{N} {\sum }_{i = 1}^{N} C h a n n e {l}_{n} ( i ) \cdot {Channel}_{m} ( i ) ;$$

Covariance between channels: Cₙcₘ_{_{cov}}:

$${c}_{n} {c}_{m} \_cov = \frac{1}{N - 1} {\sum }_{i = 1}^{N} ( C h a n n e {l}_{n} ( i ) - {c}_{n} \_avg ) \cdot ( {Channel}_{m} ( i ) - {c}_{m} \_avg ) ;$$

R_G_ratio:

$$R\_G = \frac{R - G}{R + G} ;$$

and
B_RG_ratio:

$$B\_RG = \frac{B}{R + G} .$$

III. Physiological Parameters

In some embodiments, based, at least in part, on the skin features metadata set extracted as described above, Video processing module 110a may be configured for detecting a plurality of physiological parameters, based, at least in part, on extracting a raw PPG signal form the metadata set, as illustrated by the exemplary parameter set in table 4.

TABLE 4
Physiological Parameters Set

| Feature Name | Description |
|---|---|
| PPG | The PPG signal extracted from skin pixels. |
| BPM | BPM (Bit Per Minute) signal calculation based |
| | on frequency analysis of PPG signal. |
| BPM_BL_AVG_10 | Changes in BPM over previous 10 seconds, |
| | calculated via 10 seconds overlapping time |
| | windows. |
| BPM_BL_STD_10 | Standard deviation in BMP changes over previous |
| | 10 seconds, calculated via 10 seconds overlapping |
| | time windows. |
| Resp_rate | Respiration rate signal based on frequency |
| | analysis of PPG signal. |
| Resp_BL_AVG_10 | Changes in respiration rate over previous 10 |
| | seconds, calculated via 10 seconds overlapping |
| | time windows. |
| Resp_BL_STD_10 | Standard deviation in respiration rate changes |
| | over previous 10 seconds, calculated via 10 |
| | seconds overlapping time windows. |
| HRV | Variability in BPM rate over time, detected based |
| | on detecting minimum points in the PPG signal. |

FIG. 8B schematically illustrates a process for the detection of a PPG signal in skin ROI, according to an embodiment. In some embodiments, video processing module 110a may employ one or more neural networks to detect a PPG signal in the skin metadata extracted as described above.

In some embodiments, the present invention may employ an advantageous algorithm for phase correction when estimating PPG based on a video stream. Oftentimes, in video-base PPG estimation, a matrix SKIN(h, w) of skin pixels is created, as described above, such that each cell in the matrix corresponds to a fixed position on the subject's skin. SKINₜ is the SKIN matrix in time t, such that the change in skin color over time is known for each pixel. For the most part, getting PPG signal is done using the procedure

ft(SKINₜ(h, w))→fₜ(fft)→ifft(fft).

This assumes reducing the SKIN matrix time series to a single value, and then transferring the output vector of the function from the time domain to the frequency domain, to cut out unwanted frequencies, before retransferring it back into the time domain, e.g., for further processing. This standard procedure may be flawed for video-based PPG signal extraction, because skin color changes over a specified area may appear in phases, i.e., at slightly different times. That means that reducing the SKIN matrix to a single value per a single time point can include a large amount of noise, which will be difficult to remove later on.

Accordingly, in some embodiments, the present invention provides for phase correction of the SKIN matrix as follows:

fft(SKINₜ(h, w))→fₜ(fft)→ifft(fft).

The phase correction provides first for a multi-dimensional fft on the SKIN matrix (on all the space and the time dimension), after which the reducing function may apply, to reduce all the space dimensions to a single value.

At a step 406 in FIG. 4, in some embodiments, video processing module 110a may be configured for performing PPG signal reconstruction. Remotely extracted PPG signal may contain artifacts, caused by subject movement, lighting inconsistencies, etc. In order to achieve the most accurate heart rate parameters analysis from the PPG signal, video processing module 110a may be configured for reconstructing the PPG signal, for eliminating the substandard sections. Accordingly, in some embodiments, video processing module 110a may be configured for defining sliding window of length t along the PPG signal, and detecting global minimum points in each window, from which cycle times may be derived. Then, with respect to each cycle, video processing module 110a may be configured for calculating a polynomial function which describes the current cycle, and comparing the polynomial function to a known polynomial function for a PPG signal simulation to determine which cycle's polynomial function is best fitting the known PPG polynomic function. After detecting the best fitting cycle, curve of the rest of the cycles may be adjusted by using the polynomial function of the best cycle.

In a variation on the above process, video processing module 110a may be configured for calculating an average curve of all cycles in a window. Once calculated, video processing module 110a may be configured for identifying individual cycle curves which diverge from the overall average by a specified threshold (e.g., 20-30%), wherein outliers cycles may be replaced with the average curve.

In yet another variation, video processing module 110a may be configured for extracting a set of main features from each cycle in a window, then use the PPG simulation polynomial function for estimating a hypothetical main PPG wave. Video processing module 110a then may be configured for replacing the actual curve within certain of the cycles with the hypothetical curve, based, e.g., on a threshold similarity parameter.

IV. Data Compression

In some embodiments, at a step 408 in FIG. 4, system 100 may be configured for performing data compression with respect to the extracted features. For example, in some embodiments, system 100 may perform principal component analysis (PCA) for dividing all features into common clusters.

### Tracking Based on Skin Probability Variability

In some embodiments, the present invention may employ a method for tracking of a biological object in a video image stream, based on skin classification. In some embodiments, the tacking method may be configured for segmenting each frame in the image stream, generating a classification prediction as to the probability that each segment comprises a skin segment, and then tracking a vector of the predictions over time within the image stream, to track a movement of the subject within the image stream.

In some embodiments, the tracking method disclosed herein comprises defining a series of overlapping temporal windows of duration t, wherein each window comprises a plurality of successive image frames of the video stream. Each image frame in each window may then be segmented into a plurality of segments, for example, in a 3×3 matrix. In some embodiments, other matrices, such as 9×9 may be used. The method may then be configured for extracting a skin metadata feature set of each segment in each image frame in the window, as described above under “Video Processing Methods—Feature Extraction.” A trained machine learning classifier may then be applied to the skin metadata, to generate a prediction with respect to whether a segment may be classified as human skin behavior, based, at least in part, on specified human biological patterns, such as typical human skin RGB color ranges, and typical human skin RGB color variability over time (which may be related to such parameters as blood oxygenation).

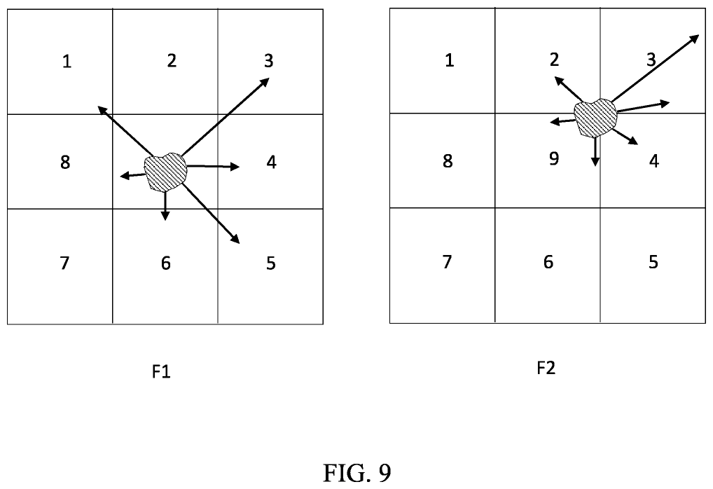

After generating all predictions for all segments in each window, the method may be configured for calculating skin prediction variability over time with respect to each segment, as the subject in the image stream shifts and moves within the image frames. Based on the calculated prediction variability, the method may derive a weighted ‘movement vector,’ which represents the movement of prediction probabilities among the segments in each frame over time. FIG. 9A illustrates a movement vector within an exemplary 3×3 matrix of segments. As can be seen, as a skin patch migrates between frames F1 and F2, segment 3 generates a next prediction in frame F2 having the highest skin classification probability. Accordingly, the movement vector in the direction of segment 3 will be assigned the highest weight. Once movement vectors are calculated for each overlapping time window, the method may derive such movement vector over the duration of the image stream.

### Inference Stage—Predicting Stress States

In some embodiments, multi-model prediction algorithm 110b may be configured for predicting stress states in a subject, based, at least in part, on a features continuously extracted from a video image stream, using the methods and processes described above under as described above under “Video Processing Methods—ROI Detection” and “Video Processing Methods—Features Extraction.” In some embodiments, the video image stream may be a real time stream. In some embodiments, the extraction process may be performed offline.

In some embodiments, multi-model prediction algorithm 110b may be configured for further predicting a state of ‘global stress’ in a human subject based, at least in part, on detecting a combination of one or more of the constituent stress categories. In some embodiments, a ‘global stress’ signal may be defined as an aggregate value of one or more individual constituent stress states in a subject. For example, a global stress value in a subject may be determined by summing the values of detected cognitive and/or emotional stress in the subject. In some variations, the aggregating may be based on a specified ratio between the individual stress categories

As noted above, in real life subject observation situations, several challenges emerge related to subject movement, lighting conditions, system latency, facial detection algorithms limitations, the quality of the obtained video, etc. For example, observed subjects may not remain in a static posture for the duration of the observation, so that, e.g., the facial region may not be fully visible at least some of the time. In another example, certain features may suffer from time lags due to system latency. For example, HRV frequency domain features consist of HF, LF and VLF spectrum ranges. Ideally, HRV analysis requires a window of at least 5 minutes. In practice, HF frequencies can become available for analysis within about 1 minute, LF within about 3 minutes, and VLF within about 5 minutes. Because HRV data is a very significant feature for predicting stress and differentiating between the different types of stress, a 1-5 minutes period of latency may be impracticable for providing real time continuous analysis.

Accordingly, the predictive model of the present invention may be configured for adapting to a variety of situations and input variables, by switching among a plurality of predictive sub-models configured for various partial-data situations. In some embodiments, multi-model prediction algorithm 110b may thus be configured for providing continuous uninterrupted real-time analytics in situations where, e.g., a facial region not continuously visible in the video stream, or in periods of data latency when not all features have come online yet.

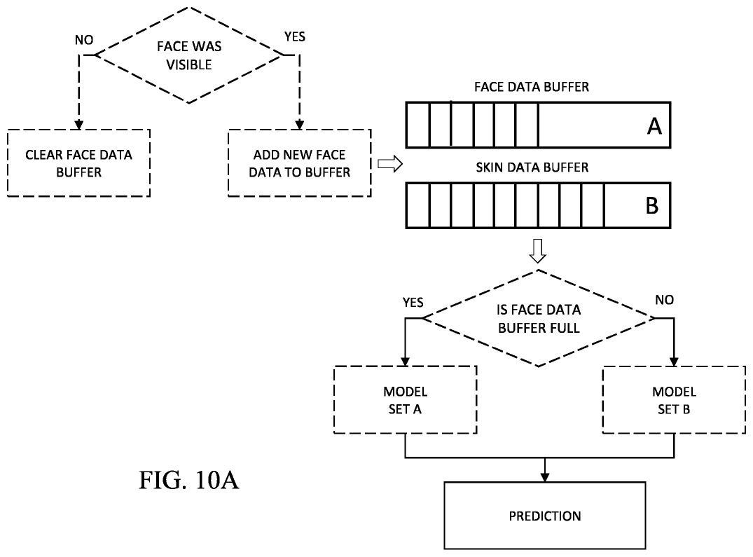

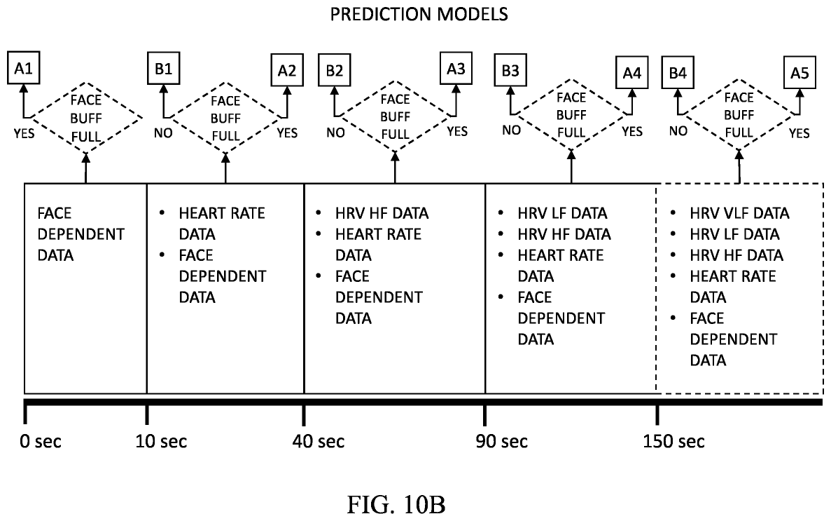

FIG. 10A schematically illustrates a model switching method according to an embodiment. Assuming a video stream of a subject where the facial region is not visible and/or not detectable in the image frames for at least part of the time, multi-model prediction algorithm 110b may be configured for switching between, e.g., the following two sets of predictive models, depending on facial region detectability:
Set A includes one or more sub-models A₁, . . . , Aₙ, each trained on a training set comprising a different combination of both facial region and skin features.
Set B includes one or more sub-models B₁, . . . , Bₙ, each trained on a training set comprising a different combination of skin features only.

In some embodiments, multi-model prediction algorithm 110b may comprise other and/or additional sub-model sets, e.g., sub-models configured for predicting stress states based on voice analysis, whole body movement analysis, and/or additional modalities.

Switching between the sets may be based, at least in part, on the time-dependent visibility of a facial region in the video stream. Within each set, switching between sub-models may be based, at least in part, on the time-dependent availability of specific features in each modality (e.g., heart rate only; heart rate and high-frequency HRV; heart rate, high-frequency HRV, and low frequency HRV; etc.).

For example, with continued reference to FIG. 10A, assuming two sliding data windows of 20 seconds each, wherein the first window includes facial region features, and the second window includes skin-related features. Each of the windows has an associated data buffer, A and B, respectively. For each period in the first window in which the facial region is not visible, all data related to that period will be removed from the relevant window, wherein periods in which the facial region is visible are pushed into buffer A. Facial features buffer A will then only get filled when there is at least a continuous 20 second window where the facial region is visible. Once skin features buffer B gets filled up, if facial features buffer A is also filled up, both overlapping buffers get merged into a single features matrix, and multi-model prediction algorithm 110b switches to using set A. If, however, facial features buffer A is empty, multi-model prediction algorithm 110b is configured for switching to using set B. Thus, multi-model prediction algorithm 110b may be configured for ensuring continuous predictive analytics, regardless of whether or not the face is visible in the image frames. In some embodiments, stress predictions based solely on set B may have an accuracy of more than 90%.

In some embodiments, multi-model prediction algorithm 110b may be configured for employing a time-dependent model-switching scheme, wherein each sub-model may be trained on a different training set comprising various features. FIG. 10B is a schematic illustration of a multi-model switching scheme, according to an embodiment. For example, skin-related features typically become available starting approximately 10 seconds after the beginning of the analytical time series. Thus, in the first 10 seconds of the analytical time series, only facial features may be available (assuming the facial region is detectable in the image stream), and only set A models may be applied.

In a subsequent period, e.g., from 10 to 40 seconds, skin-related heart-rate features, such as heart rate data, may come online and may be used for prediction, with or without facial features (depending on availability). Accordingly, multi-model prediction algorithm 110b may then switch to sub-models A2 or B1, respectively.

In a subsequent period, e.g., from 40 to 90 seconds, HF HRV features may further become available, again, with or without facial features. Accordingly, multi-model prediction algorithm 110b may then switch to sub-models A3 or B2, respectively.

In a subsequent period, e.g., from 90 to 150 seconds, LF HRV features may further become available, again, with or without facial features. Accordingly, multi-model prediction algorithm 110b may then switch to sub-models A4 or B3, respectively.

From 150 seconds onward, VLF HRV features may be observed, again, with or without facial features. Accordingly, multi-model prediction algorithm 110b may then switch to sub-models A5 or B4, respectively.

In some embodiments, with each progression of sub-models, better prediction accuracy may be expected.

In some embodiments, multi-model prediction algorithm 110b may be further configured for detecting a significant response (SR) state in a subject, which may be defined as consistent, significant, and timely physiological responses in a subject, in connection with responding to a relevant trigger (such as a test question, an image, etc.). In some embodiments, detecting an SR state in a subject may indicate an intention on part of the subject to provide a false or deceptive answer to the relevant test question.

In some embodiments, an SR state may be determined based, at least in part, on one or more predicted stress states and/or a predicted states of global stress in the subject. In some embodiments, multi-model prediction algorithm 110b may be configured for calculating an SR score based, at least in part, on a predicted global stress signal with respect to a subject. For example, the SR score may be equal to an integral of the global stress signal taken over an analysis window, relative to a baseline value. In some embodiments, multi-model prediction algorithm 110b may be configured for calculating an absolute value of the change in global stress signal from the baseline, based on the observation that, in different subjects, SR may be expressed variously as increasing or decreasing (relief) trends of the global stress signal. In other embodiments, SR detection may be further based on additional and/or other statistical calculations with respect to each analysis window, or segments of an analysis window. Such statistical calculations may include, but are not limited to, mean values of the various segments within an analysis window, standard deviation among segments, and/or maximum value and minimum value within an analysis window.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a hardware processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowcharts and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

1. A system comprising:
at least one hardware processor; and
a non-transitory computer-readable storage medium having stored thereon program instructions, the program instructions executable by the at least one hardware processor to:
receive, as input, a video image stream of a bodily region of a subject,
continuously extract from said video image stream at least some of:
(i) facial parameters of said subject,
(ii) skin-related features of said subject, and
(iii) physiological parameters of said subject, and
apply a first trained machine learning classifier selected from a group of trained machine learning classifiers, based, at least in part, on a detected combination of said facial parameters, skin-related features, and physiological parameters, to determine one or more states of stress in said subject,
wherein said group of trained machine learning classifiers comprises a hierarchical cascade of machine learning classifiers.

2. The system of claim 1, wherein said bodily region is at least one bodily region selected from a group consisting of: whole body, facial region, and one or more skin regions.

3. (canceled)

4. The system of claim 1, wherein said applying further comprises selecting a next machine learning classifier for application, from said group of trained machine learning classifiers, based, at least in part, on detecting time-dependent changes in said detected combination of said facial parameters, skin-related features, and physiological parameters.

5. The system of claim 1, wherein said applying comprises selecting a number of machine learning classifiers from said group, and wherein said determining is based, at least in part, on a combination of determinations by each of said classifiers.

6. The system of claim 1, wherein at least one of said machine learning classifiers in said group of trained machine learning classifiers is trained on a training set comprising only one of physiological parameters, skin-related features, and physiological parameters.

7. The system of claim 1, wherein at least one of said machine learning classifiers in said group of trained machine learning classifiers is trained on a training set comprising a combination of two or more of physiological parameters, skin-related features, and physiological parameters.

8. (canceled)

9. (canceled)

10. (canceled)

11. The system of claim 1, wherein said determining further comprise detecting a state of global stress in said subject, based, at least in part, on said determined one or more states of stress in said subject, wherein said states of stress are selected from the group consisting of: neutral stress, cognitive stress, positive emotional stress, and negative emotional stress.

12. The system of claim 1, wherein said plurality of physiological parameters comprise at least some of a photoplethysmogram (PPG) signal, heartbeat rate, heartbeat variability (HRV), respiration rate, and respiration variability.

13. The system of claim 1, wherein said plurality of skin-related features represent time-dependent spectral reflectance intensity from a skin region of said subject, and wherein said skin-related features are based, at least in part, on image data values in said video image stream, in at least one color representation model selected from the group consisting of: RGB (red-green-blue), HSL (hue, saturation, lightness), HSV (hue, saturation, value), and YCbCr.

14. (canceled)

15. The system of claim 1, wherein said plurality of facial parameters comprise at least some of: eye blinking patterns, eye movement patterns, and pupil movement patterns,
wherein said eye blinking patterns comprise at least some of: changes in eye aspect ratio, duration between successive eyelid closures, duration of eye closure, duration of eye opening, eye blinking rate, and eye blinking rate variability, and wherein said pupil movements comprise at least some of pupil coordinates change, pupil movement along X-Y axes, acceleration of pupil movement along X-Y axes, and pupil movement relative to eye center.

16. (canceled)

17. (canceled)

18. A method comprising:
receiving, as input, a video image stream of a bodily region of a subject;
continuously extracting from said video image stream at least some of:
(i) facial parameters of said subject,
(ii) skin-related features of said subject, and
(iii) physiological parameters of said subject; and
applying a first trained machine learning classifier selected from a group of trained machine learning classifiers, based, at least in part, on a detected combination of said facial parameters, skin-related features, and physiological parameters, to determine one or more states of stress in said subject, wherein said group of trained machine learning classifiers comprises a hierarchical cascade of machine learning classifiers.

19. The method of claim 18, wherein said bodily region is at least selected from the group consisting of: whole body, facial region, and one or more skin regions.

20. (canceled)

21. The method of claim 18, wherein said applying further comprises selecting a next machine learning classifier for application, from said group of trained machine learning classifiers, based, at least in part, on detecting time-dependent changes in said detected combination of said facial parameters, skin-related features, and physiological parameters.

22. The method of claim 18, wherein said applying comprises selecting a number of machine learning classifiers from said group, and wherein said determining is based, at least in part, on a combination of determinations by each of said classifiers.

23. The method of claim 18, wherein at least one of said machine learning classifiers in said group of trained machine learning classifiers is trained on a training set comprising only one of physiological parameters, skin-related features, and physiological parameters.

24. The method of claim 18, wherein at least one of said machine learning classifiers in said group of trained machine learning classifiers is trained on a training set comprising a combination of two or more of physiological parameters, skin-related features, and physiological parameters.

25. (canceled)

26. (canceled)

27. (canceled)

28. The method of claim 18, wherein said determining further comprise detecting a state of global stress in said subject, based, at least in part, on said determined one or more states of stress in said subject, and wherein said states of stress are selected from a group consisting of: neutral stress, cognitive stress, positive emotional stress, and negative emotional stress.

29. The method of claim 18, wherein said plurality of physiological parameters comprise at least some of a photoplethysmogram (PPG) signal, heartbeat rate, heartbeat variability (HRV), respiration rate, and respiration variability.

30. The method of claim 18, wherein said plurality of skin-related features represent time-dependent spectral reflectance intensity from a skin region of said subject, and wherein said skin-related features are based, at least in part, on image data values in said video image stream, in at least one color representation model selected from the group consisting of: RGB (red-green-blue), HSL (hue, saturation, lightness), HSV (hue, saturation, value), and YCbCr.

31. (canceled)

32. The method of claim 18, wherein said plurality of facial parameters comprise at least some of: eye blinking patterns, eye movement patterns, and pupil movement patterns,
wherein said eye blinking patterns comprise at least some of: changes in eye aspect ratio, duration between successive eyelid closures, duration of eye closure, duration of eye opening, eye blinking rate, and eye blinking rate variability, and
wherein said pupil movements comprise at least some of pupil coordinates change, pupil movement along X-Y axes, acceleration of pupil movement along X-Y axes, and pupil movement relative to eye center.

33-51. (canceled)
